(19) United States
(12) Patent Application Publication
(10) Pub. No.: US 2014/0275909 A1
(43) Pub. Date: Sep. 18, 2014

(54) CATHETER AND SHEATH INTRODUCER

(71) Applicant: Lightship Medical Limited, London (GB)

(72) Inventors: Mithun NARAYANAN, Oxfordshire (GB); Robert PERKINS, Oxfordshire (GB); Roger PAMMENT, Oxfordshire (GB)

(73) Assignee: Lightship Medical Limited, London (GB)

(21) Appl. No.: 14/213,177

(22) Filed: Mar. 14, 2014

(30) Foreign Application Priority Data
Mar. 15, 2013 (GB) 1304807.9

(51) Int. Cl. A61B 5/145; A61B 5/00

(52) U.S. Cl. CPC A61B 5/14532; A61B 5/6852
USPC 600/365

(57) ABSTRACT

The present disclosure provides a catheter comprising one or more infusion lumens each terminating at an infusion eye, and one or more sensor lumens each terminating at a sensor eye. The present disclosure also provides a sheath introducer comprising one or more sensor lumens each terminating at a sensor eye, and an introducer lumen terminating at an introducer eye.

### FIELD

The present disclosure relates to catheters, sheath introducers, kits including catheters, kits including sheath introducers, methods of using catheters and methods of using sheath introducers. The catheter or sheath introducer can be used with an invasive sensor.

### BACKGROUND

Invasive biosensors, and in particular intravenous biosensors, are known in the art and include those intended for use in intensive care environments. However, care must be exercised in the placement of the sensor. If the sensor is located within a vein into which, as is common in intensive care environments, fluids are being infused, the biological parameters which the sensor attempts to measure can be influenced. For example, in the case of an invasive glucose sensor, the glucose level detected by the sensor can be artificially high if glucose solution is being infused into the same vein. Alternatively, detected blood glucose can be artificially low if the blood is diluted by the infusion of a non glucose-containing fluid. For these reasons it has previously been necessary to place invasive biosensors at a location remote from sites at which fluids are infused. This creates a burden on patients and medical staff as intravenous devices must be inserted into the patient at multiple sites.

Further difficulties in placing an invasive biosensor into a vein arise in cases where a subject (e.g. a Coronary Artery Bypass Graft or CABG patient) also requires a separate catheter (e.g. a Pulmonary Artery or Swan Ganz Catheter (PAC)) to be placed into a vein, and particularly in cases where both the invasive biosensor and the separate catheter would ideally be placed in the same vein, e.g. the right internal jugular. In these cases it would be possible to introduce both the invasive biosensor and the separate catheter into the same vein by “double sticking” or making two insertions at different locations in the same vein. However, double sticking carries an increased risk of infection. It would also be possible to insert each device into a separate vein, e.g. to insert a PAC in the right internal jugular, and an invasive biosensor in another vessel. However, this method would be puncturing yet another vein thereby raising the risk of device complication and infection.

### BRIEF SUMMARY

The catheter and method provided herein have the advantage of minimising the burden on patients and medical staff by allowing an invasive biosensor to be placed within the same device used for the infusion of fluids, while enabling accurate measurement of the parameter the sensor seeks to measure. The present disclosure therefore provides a catheter comprising one or more infusion lumens each terminating at an infusion eye, and one or more sensor lumens each terminating at a sensor eye. The advantages described above are typically achieved by a multi-lumen catheter in which a sensor eye/s is located upstream of the fluid eye/s.

Many patients (e.g intensive care patients) are routinely fitted with a sheath introducer in the right internal jugular vein in order to allow for insertion of a catheter (e.g. a PAC) as and when required during treatment and/or diagnosis of the patient. However, routine placement of the sheath introducer means that separate placement of the catheter provided herein would either require double sticking the vein carrying the sheath introducer, or puncturing another vein. These difficulties are addressed herein by providing an integrated sheath introducer allowing an invasive biosensor and a central venous catheter to be placed in the same vein without making multiple insertions at different locations in the vein (i.e. without “double sticking”), and without requiring a physician to deviate significantly from the current practice of routinely fitting patients with a sheath introducer. The present disclosure therefore provides a sheath introducer comprising one or more sensor lumens each terminating at a sensor eye, and an introducer lumen terminating at an introducer eye.

The present disclosure provides a method of inserting an invasive biosensor into a vein through a catheter comprising one or more infusion lumens each terminating at an infusion eye, and one or more sensor lumens each terminating at a sensor eye, the catheter being located in a vein, the method comprising inserting the biosensor into the vein through a sensor lumen of the catheter. The method may comprise an initial step of inserting the catheter into a vein.

The present disclosure also provides a method of inserting an invasive biosensor into a vein through a sheath introducer comprising an introducer lumen terminating at an introducer eye, and a sensor lumen terminating at a sensor eye, the introducer sheath being located in a vein, the method comprising inserting the biosensor into the vein through a sensor lumen of the introducer sheath. The method may comprise an initial step of inserting the sheath introducer into a vein.

The present disclosure also provides a kit comprising a catheter according to the present disclosure and an invasive biosensor. The present disclosure also provides a kit comprising a sheath introducer according to the present disclosure and an invasive biosensor. In some cases the invasive biosensor is a glucose sensor, such as the sensors disclosed in US 2009/018418 and WO 2012/095628.

### BRIEF DESCRIPTION OF THE DRAWINGS

FIG. 1 shows a catheter according to the present disclosure.

FIG. 2 shows a cross-section of the multi-lumen shaft of the catheter of the present disclosure.

FIG. 3 shows the distal portion of the multi-lumen shaft of the catheter according to the present disclosure.

FIG. 4 shows the distal portion of the multi-lumen shaft of the catheter according to the present disclosure when in use with a biosensor.

FIG. 5 shows a portion of a catheter according to the present disclosure relative to a patient's body when in use.

FIG. 6 shows a distal portion of a sheath introducer according to the present disclosure when in use with a biosensor.

FIG. 7 shows a sheath introducer according to the present disclosure

FIG. 8 shows a cross-section of a sheath introducer of the present disclosure.

### DETAILED DESCRIPTION

The present disclosure provides a catheter comprising one or more infusion lumens each terminating at an infusion eye, and one or more sensor lumens each terminating at a sensor eye.

As used herein the term upstream refers to a direction on the catheter which is further upstream in the flow of blood in the vein when the catheter is in use, i.e. along the catheter moving away from the distal end. As used herein the term downstream refers to a direction on the catheter which is further downstream in the flow of blood in the vein when the catheter is in use, i.e. moving from the proximal end towards the distal end of the catheter.

The catheter is configured to be inserted into a vein so that the distal end of the catheter is downstream of the point of insertion. One infusion lumen typically terminates at a distal infusion eye which is located at the distal end of the catheter. The one or more sensor eyes are typically located upstream of the distal infusion eye. When a plurality of infusion lumens are present, additional infusion lumens typically terminate at infusion eyes located upstream of the distal infusion eye and downstream of the one or more sensor eyes. The totality of the one or more sensor eyes are typically located upstream of the totality of the infusion eyes.

The separation between the one or more sensor eyes and one or more infusion eyes is typically sufficient to avoid fluid infusion interfering with sensor operation even in the event that reflux of the infused fluid takes place. Reflux is an effect whereby some of a fluid infused through an infusion eye travels a short distance upstream of the infusion eye. The or each sensor eye is therefore typically located at least 40 mm or more, e.g. 60 mm or more, from each infusion eye.

The one or more infusion lumens are typically any lumen suitable for intravenous infusion of a fluid but may not be suitable for carrying a sensor. The one or more sensor lumens are typically any lumen suitable for carrying an invasive biosensor, and may also be suitable for intravenous infusion of a fluid, e.g. at a low rate of flow or at a high rate of flow.

The factors determining whether a lumen is suitable for infusing a fluid or for carrying an invasive biosensor or both include, for example, the dimensions of the lumen and the eye at which it terminates, and the location on the catheter of the eye at which it terminates relative to the location of other eyes. The infusion lumen terminating at the distal eye is typically of greater diameter than any additional infusion lumens present. The one or more sensor lumens are typically of greater diameter than any additional infusion lumens. Thus, a sensor lumen typically has a diameter large enough to accept an invasive biosensor (typically 1.15 mm or more, e.g. 1.15 mm to 1.25 mm, more typically 1.18 mm to 1.22 mm) Sensor lumens having larger diameters are however contemplated, e.g. 1.20 mm or more, 1.25 mm or more, 1.30 mm or more or 1.35 mm or more. Sensor lumens having smaller diameters are also contemplated, e.g. 0.80 mm or more, or 0.90 mm or more. An infusion lumen terminating at a distal eye typically has a diameter comparable to that of a sensor lumen as described above to allow use of a guide wire to assist catheter placement. Thus, the diameter of a lumen terminating at a distal eye is typically greater than 1.0 mm, e.g. 1.05 mm to 1.25 mm, typically 1.05 mm to 1.15 mm. Any additional infusion lumen typically has a narrower diameter and may be too small to accept an invasive biosensor (typically less than 1.0 mm, e.g. 0.7 mm to 0.9 mm, more typically 0.75 mm to 0.85 mm)

Further, a lumen will typically be unsuitable for carrying an invasive biosensor (and can therefore be considered an infusion lumen) if it terminates at an eye which is downstream of an infusion eye (e.g. an eye at which terminates a lumen having a diameter of less than 1.0 mm) Thus the lumen terminating at the distal eye is typically an infusion lumen and not a sensor lumen. A lumen will also typically be unsuitable for carrying an invasive biosensor (and can therefore be considered an infusion lumen) if it terminates at an eye which is less than 65 mm, e.g. less than 40 mm, upstream of an infusion eye (i.e. a distal eye or an eye at which terminates a lumen having a diameter of less than 1.15 mm)

The present disclosure also provides a sheath introducer comprising one or more sensor lumens each terminating at a sensor eye, and an introducer lumen terminating at an introducer eye

A sheath introducer is typically a device capable of being partially placed into a subject's vein, with a distal portion located in the subject's vein and a proximal portion external to the subject's body, and wherein the sheath introducer is capable of accepting an additional device (e.g. a PAC). The sheath introducer is typically configured to enable the additional device to be inserted at the proximal portion of the sheath introducer into an introducer lumen, and extend through the introducer lumen and through an introducer eye at which the introducer lumen terminates, so as to project into the subject's vein.

The sheath introducer is configured to be inserted into a vein so that the distal end of the sheath introducer is downstream of the point of insertion. The introducer lumen typically terminates at a distal introducer eye which is located at the distal end of the sheath introducer. The one or more sensor eyes are typically located upstream of the distal introducer eye.

The one or more sensor eye is located sufficiently upstream of the introducer eye that when in use, a sensing region of a sensor carried by the sensor lumen is a sufficient distance upstream of an eye of any catheter that may extend from the introducer eye, through which catheter a fluid may be infused, for the sensor reading to be unaffected any infusion of any said fluid. The or each sensor eye is therefore typically located 30 mm or more, more typically 40 mm or more, e.g. 60 mm or more, typically about 70 mm upstream of the introducer eye. The fluid may be infused at a rate of 100 ml/min or more, e.g. 150 ml/min. Fluid may also be infused through the sensor lumen of the sheath introducer. However the rate of infusion of fluid through the sensor lumen is typically low enough not to affect the sensor reading, and may for example be 4 ml/hour or less.

The one or more sensor lumens are typically as described above with respect to the catheter of the present disclosure, and may be any lumen suitable for carrying a sensor. The one or more sensor lumens typically have a cross section which is not substantially circular. Typically the smallest cross-sectional dimension of the one or more sensor lumens is about 1.15 mm or more, e.g about 1.15 mm to about 1.75 mm, typically about 1.50 mm. Typically, the one or more sensor lumens are not suitable for carrying a catheter.

The factors determining whether a lumen of a sheath introducer is suitable for carrying a catheter include, for example, the dimensions of the lumen and the eye at which it terminates, and the location on the sheath introducer of the eye at which it terminates relative to the location of other eyes. The introducer lumen terminating at the distal eye is typically of greater diameter than the one or more sensor lumens. An introducer lumen terminating at a distal eye typically has a diameter greater than that of a sensor lumen as described above to allow catheter placement. Thus, the diameter of an introducer lumen terminating at a distal eye is typically greater than 2.00 mm, e.g. 2.00 mm to 3.50 mm, more typically 2.50 mm to 3.00 mm.

The effective length of the sheath introducer is typically such that it does not damage the patient's vasculature when in use, e.g. such that it does not touch the patient's tricuspid valve when inserted in the right internal jugular. The sheath introducer of the present disclosure typically has an effective length not more than 13 cm, e.g. 12 to 13 cm or 12.5 to 13 cm. The effective length is the distance from the distal end of the sheath introducer to the furthest point which can be inserted into a patient's vein, typically the distance between the distal end of the sheath introducer and a collar of the sheath introducer.

The sheath introducer typically further comprises a side port connected to the introducer lumen. Fluid can be infused (e.g. at a low rate of flow or a high rate of flow) through the introducer lumen to the introducer line via the side port.

The present disclosure will now be further described, by way of non-limitative example only, with reference to the accompanying schematic drawings.

A catheter 1 according to the present disclosure comprises a multi-lumen shaft 6 which is substantially circular in cross section, although others e.g. elliptical cross sections are also envisaged. The outer body can be made from any material used for the outer body of catheters known in the art.

The catheter 1 has an infusion lumen 4c which terminates at a distal infusion eye 2a at its distal end and may have additional infusion lumens 4a terminating at additional infusion eyes 2b located upstream of the distal infusion eye 2a. When additional infusion eyes 2b are present the total number of infusion eyes present is typically any number seen in multiple lumen infusion-only catheters known in the art, e.g. 2, 3, 4 or 5. The catheter has a sensor lumen 4b terminating at a sensor eye 3. The sensor eye 3 is located upstream of the distal infusion eye 2a and additional infusion eyes 2b. Although not depicted in the illustrated embodiment, catheters having multiple sensor lumens and multiple sensor eyes are also envisaged.

The furthest downstream of the sensor eyes 3 is typically located 30 mm or more, e.g. 40 mm or more, 50 mm or more, 60 mm or more, 70 mm or more, or 80 mm or more, typically 90 mm or more, upstream of the distal end of the catheter.

The furthest upstream of the infusion eyes 2 is typically located 40 mm or more, e.g. 65 mm or more downstream of the nearest sensor eye 3.

Each sensor lumen and infusion lumen may independently have a substantially circular cross section. The one or more sensor lumens typically have a diameter of about 1.15 mm or more, e.g about 1.15 mm to about 1.25 mm, typically about 1.20 mm. The diameter of the lumen 4c terminating at the distal infusion eye 2a is typically greater than 1.00 mm and less than 1.15 mm, e.g. 1.05 mm to 1.12 mm, typically about 1.10 mm for a typical guide wire. The one or more additional infusion lumens 4a typically have a diameter of 1.0 mm or less, e.g. about 0.70 mm to about 0.90 mm, typically about 0.80 mm.

Alternatively each sensor lumen and infusion lumen may independently have a cross section which is not substantially circular. When a sensor lumen does not have a substantially circular cross section, its smallest cross-sectional dimension is typically as described above with respect to the diameter of a substantially-circular sectioned sensor lumen. When an infusion lumen does not have a substantially circular cross section, its smallest cross-sectional dimension is typically as described above with respect to the diameter of a substantially circular sectioned infusion lumen.

In use, a fluid (e.g. saline) will typically be passed though the sensor lumen 4b at a low rate of flow, typically at a rate of up to about 4 ml/hr, e.g. about 1 ml/hr, in order to avoid blood entering the sensor eye 3 (lumen 4b) where it could coagulate. When an infusion eye 2a or 2b is used, a fluid is typically infused through an infusion lumen 4a or 4c at a high rate of flow, e.g. 100 ml/min or more, e.g. 150 ml/min. One or more, for example, all, of the infusion eyes may be used for high flux fluid. Alternatively, fluid (e.g. saline) may be infused at a low rate of flow through one or more infusion eyes. Such low flow helps to avoid blood entering the catheter where it could coagulate. Said fluid (e.g. saline) will typically be passed though the infusion eye 2a or 2b at a rate of up to about 4 ml/hr, e.g. 0.5 to 2 ml/hr.

In the method of the present disclosure, the catheter 1 may comprise one or more fluids which are in flux along one of more infusion lumens 4a or 4c of the catheter 1. One or more fluids are typically infused through one or more infusion lumens 4a or 4c of the catheter 1, typically at a high rate of flow, e.g. 100 ml/min or more, typically 100 ml/min to 200 ml/min, more typically 125 to 175 ml/min.

In the method of the present disclosure a fluid may be infused through the sensor eye 3 (lumen 4b) at a low rate of flow, e.g. 4 ml/hr or less, typically 0.5 to 2 ml/hr. In order to avoid the sensor 9 being affected by the fluid passed through a sensor lumen 4b and out of sensor eye 3, the sensor 9 is typically passed through a sensor lumen 4b so that the sensor 9 protrudes from the sensor eye 3 and a sensing region 10 of the sensor 9 is located 15 mm or more (e.g. 20 mm or more, typically 30 mm) downstream of said sensor eye 3. The sensing region 10 is the part of the sensor which carries out the sensing, e.g. the part of the sensor which contains any sensing indicators. The sensor 9 and sensing region 10 of the sensor 9 are, for example, as described in US 2009/018418 and WO 2012/095628.

The proximal end of the catheter multi-lumen shaft 6 terminates at a collar 5 which is suitable for securing the catheter to a patient's skin while in use. In one embodiment the collar 5 is triangular, trapezoidal or wedge shaped in section so that the collar will only sit comfortably on the patient's body in a desired orientation. In other words the collar 5 typically has a flat base when viewed in cross section. The collar may be secured to the patient's skin by a securing means located at the base of the collar, thereby further helping to orient the catheter 1 relative to the patient's body 13. In this embodiment, with the catheter 1 oriented relative to the patient's body 13, the sensor eye 3 may be configured so as to be on a side of the catheter opposed to the securing means, which is located against the patient's skin. The sensing region 10 of the sensor 9 may thereby be positioned away from the wall of the vein 14. The positioning of the sensing region in this way is determined by the tendency of the catheter to lie against the wall of the vein furthest from the patient's skin, as illustrated in FIG. 5.

Lumens terminating upstream of the distal end of the catheter may terminate by having a portion of adhesive blocking the lumen distal of the eye. The sensor lumen may have a ramped configuration where it meets the sensor eye to enable a smooth transition into the vein. Thus, the sensor lumen typically terminates in a ramp where it meets the sensor eye. The ramped configuration may be formed by shaping the adhesive at the terminus of the sensor lumen. The adhesive blocking an infusion lumen at its terminus typically does not have a ramped configuration, although infusion lumens having a ramped configuration at the terminus are contemplated.

The proximal end of each infusion lumen 4a and 4c terminates at an adaptor 8 suitable for receiving a fluid to be infused and/or suitable for extracting sampled blood into a suitable receptacle. Each infusion lumen 4a and 4c may be clamped with a clamp 12 between the collar 5 and adaptor 8. The proximal end of the sensor lumen 4b terminates at an adaptor suitable for connecting the sensor lumen 4b and sensor 9 to a monitor. The sensor lumen adaptor is terminated with a suitable means that will not cause damage to the sensor, e.g. a cap.

The distal end of the catheter may be profiled for ease of insertion. The distal end of the catheter may be softer and/or more flexible than the main body of the catheter for ease of insertion. In an embodiment one of the infusion eyes is a sampling eye 2b′. The sampling eye 2b′ is typically located 20 mm or more (e.g. 20 mm to 40 mm, typically about 25 mm) upstream of the nearest other infusion eye 2b, and 40 mm or more (e.g. 40 mm to 60 mm, typically 40 mm to 50 mm) downstream of the nearest sensor eye 3. When a sampling eye 2b′ is present each sensor eye 3 is typically located 40 mm or more upstream of the sampling eye 2b′, and 65 mm or more upstream of the infusion eyes 2a and 2b. The sampling eye 2b′ and lumen 4c terminating at the sampling eye 2b′ may be used to sample blood from a vein e.g. during sensor quality control procedures, or if a blood sample is required for any other reason.

The sampling eye 2b′ may be used as an infusion eye during normal clinical use. If however, a sampling eye 2b′ is not used either for sampling or for infusion at a high rate of flow then a fluid (e.g. saline) is infused at a low rate of flow as described above.

An embodiment of the sheath introducer of the present disclosure is describe with reference to FIGS. 6, 7 and 8. A sheath introducer 100 according to the present disclosure comprises a shaft 600, an introducer lumen 400a terminating at a distal introducer eye 200, a sensor lumen 400b terminating at a sensor eye 300, and a side port 400c connecting with the introducer lumen 400a.

The shaft 600 is substantially circular in cross section, although others e.g. elliptical cross sections are also envisaged. The shaft tapers to the distal introducer eye 200 at the distal end of the sheath introducer. The outer body can be made from any material used for the outer body of sheath introducers known in the art. The distal end of the sheath introducer may be softer and/or more flexible than the main body of the sheath introducer for ease of insertion.

The introducer lumen 400a terminates at a distal introducer eye 200 at its distal end. The sensor lumen 400b terminates at a sensor eye 300. The sensor eye 300 is located sufficiently upstream of the distal introducer eye 200 that when in use, a sensing region of a sensor carried by the sensor lumen is a sufficient distance upstream of any eye of a catheter extending from the distal introducer eye 200 from which a fluid is infused for the sensor reading to be unaffected by infusion of said fluid. The sensor eye 300 is typically located 30 mm or more upstream of the distal introducer eye 200, more typically 40 mm or more, 50 mm or more, 60 mm or more, or about 70 mm (e.g 65-75 mm) upstream of the distal introducer eye 200.

The introducer lumen 400a has a substantially circular cross section. The diameter of the introducer lumen 400c is typically greater than 2.00 mm, e.g. 2.00 mm to 3.50 mm, more typically 2.50 mm to 3.00 mm.

The proximal end of the introducer lumen 400a terminates at a hemostasis valve 800a used to seal off the introducer lumen when not carrying a catheter, and aid prevention of blood loss and/or air embolism.

The sensor lumen 400b has a cross section which is not substantially circular. To enable the sensor lumen 400b to carry a sensor 9 the smallest cross-sectional dimension of the sensor lumen 400b is typically about 1.15 mm or more, e.g. about 1.15 mm to about 1.75 mm, more typically about 1.50 mm (e.g. 1.45 to 1.55 mm)

The proximal end of the sensor lumen 400b terminates at an adaptor suitable for connecting the sensor lumen 400b and sensor 9 to a monitor. The sensor lumen adaptor is terminated with a suitable means that will not cause damage to the sensor, e.g. a cap.

The side port 400c may have a substantially circular cross section. The side port 400c may have a diameter greater than 2.00 mm, e.g. 2.50 mm to 3.50 mm, typically 2.75 mm to 3.25 mm. Alternatively the side port 400c may have a cross section which is not substantially circular. When the side port 400c does not have a substantially circular cross section, its smallest cross-sectional dimension is typically as described above with respect to the diameter of a substantially-circular sectioned side port.

The proximal end of the side port 400c terminates at an adaptor 800c suitable for receiving a fluid to be infused and/or suitable for extracting sampled blood into a suitable receptacle.

In use, a fluid (e.g. saline) will typically be passed though the sensor lumen 400b at a low rate of flow, typically at a rate of up to about 4 ml/hr, typically 0.5 to 2 ml/hr e.g. about 1 ml/hr, in order to avoid blood entering the sensor eye 300 where it could coagulate. A fluid may be infused through the introducer lumen 400a via the side port 400c. Fluid may be infused through the introducer lumen at a high rate of flow, e.g. 100 ml/min or more, e.g. 150 ml/min, or at a low rate of flow of up to about 4 ml/hr, e.g. about 1 ml/hr.

In order to avoid the sensor 9 being affected by fluid passed through a sensor lumen 400b and out of sensor eye 300, in the method of the present disclosure the sensor 9 is typically passed through a sensor lumen 400b so that the sensor 9 protrudes from the sensor eye 300 such that a sensing region 10 of the sensor 9 is located 15 mm or more (e.g. 20 mm or more, typically 30 mm) downstream of said sensor eye 300. The sensing region 10 is the part of the sensor which carries out the sensing, e.g. the part of the sensor which contains any sensing indicators. The sensor 9 and sensing region 10 of the sensor 9 are, for example, as described in US 2009/018418 and WO 2012/095628.

The proximal portion of the sheath introducer shaft 600 terminates at a collar 500 which is suitable for securing the catheter to a patient's skin while in use. In one embodiment the collar 500 is as described above with respect to the catheter of the disclosure.

The sensor lumen 400b may have a ramped configuration as described above with respect to the catheter of the disclosure.

The disclosure of all documents referred to herein is hereby incorporated by reference in its entirety. The foregoing description illustrates the disclosure by way of example and not by way of limitation. The present disclosure includes embodiments other than those illustrated, within the scope of the claims.

1. A catheter comprising one or more infusion lumens each terminating at an infusion eye, and one or more sensor lumens each terminating at a sensor eye.

2. A catheter according to claim 1 which is configured to be inserted into a vein so that the distal end of the catheter is downstream of the point of insertion, and wherein the one or more sensor eyes are upstream of the one or more infusion eyes.

3. A catheter according to claim 1 wherein the one or more sensor eyes are located 30 mm or more from the distal end of the catheter.

4. A catheter according to claim 1 wherein the one or more sensor eyes are located 60 mm or more from the distal end of the catheter.

5. A catheter according to claim 1 wherein the one or more sensor eyes are located 90 mm or more from the distal end of the catheter.

6. A catheter according to claim 1 wherein each sensor eye is located 40 mm or more from each infusion eye.

7. A catheter according to claim 1 wherein each sensor eye is located 65 mm or more from each infusion eye.

8. A catheter according claim 1 wherein the one or more sensor lumens have a diameter of about 1.15 mm or more.

9. A catheter according to claim 1 wherein the catheter comprises an infusion lumen terminating at a distal eye and optionally one or more additional infusion lumens, which additional infusion lumens have a diameter of 1.00 mm or less.

10. A method for inserting an invasive biosensor into a vein through a catheter comprising one or more infusion lumens each terminating at an infusion eye, and one or more sensor lumens each terminating at a sensor eye, the method comprising inserting the catheter into the vein and inserting the biosensor into the vein through a sensor lumen of the catheter.

11. A method according to claim 10 wherein the method further comprises infusing one or more fluids through one or more infusion lumens of the catheter.

12. A method according to claim 10 wherein the catheter comprises one or more fluids which are in flux along one of more infusion lumens of the catheter.

13. A method according to claim 10 wherein the sensor lumen is 65 mm or more upstream of the infusion lumen through which fluid is infused.

14. A method according to claim 10 wherein the invasive biosensor is inserted into the vein so that a sensing region of the invasive biosensor is 20 mm or more downstream of the sensor eye.

15. A method according to claim 10 wherein a fluid is infused through the sensor lumen at a rate of up to 4 ml/hr and the fluid infused through the infusion lumen is infused at a rate greater than 4 ml/hr.

16. A method according claim 10 wherein the catheter is as defined in any one of claims 1 to 9

17. A kit comprising a catheter according to claim 1 and an invasive biosensor.

18. A sheath introducer comprising one or more sensor lumens each terminating at a sensor eye, and an introducer lumen terminating at an introducer eye.

19. A sheath introducer according to claim 18 wherein the one or more sensor eyes are located 30 mm or more from the distal end of the catheter.

20. A sheath introducer according to claim 18 wherein the one or more sensor eyes are located 60 mm or more from the distal end of the catheter.

21. A method for inserting an invasive biosensor into a vein through a sheath introducer comprising an introducer lumen terminating at an introducer eye, and one or more sensor lumens each terminating at a sensor eye, the method comprising inserting the sheath introducer into the vein, and inserting the biosensor into the vein through a sensor lumen of the sheath introducer.

22. A method according to claim 21 wherein a catheter is present in the introducer lumen, a fluid is infused through the catheter at a rate of 100 ml/min or more, and a fluid is infused through a sensor lumen of the sheath introducer at a rate of 4 ml/hr or less.

23. A kit comprising a sheath introducer according to claim 18 and an invasive biosensor.
